(19) United States
(12) Patent Application Publication
(10) Pub. No.: US 2013/0004599 A1
(43) Pub. Date: Jan. 3, 2013

(54) FORMULATION FOR TRANSDERMAL DELIVERY TO PROMOTE SLEEP

(75) Inventor: Sam SCHWARTZ, Woodland Hills, CA (US)

(73) Assignee: MASADA HEALTH & BEAUTY CORPORATION, Chatsworth, CA (US)

(21) Appl. No.: 13/173,693

(22) Filed: Jun. 30, 2011

(51) Int. Cl. A61K 36/84; A61P 25/00

(52) U.S. Cl. 424/733

(57) ABSTRACT

A topically administered composition to promote sleep, comprising Valeriana officinalis, alpha lipoic acid, at least one natural herb, Dead Sea mineral salts, and, a cream or gel carrier material suitable for topical application.

### FIELD

The present disclosure relates to topical creams, and, more particularly, a topical cream to promote sleep.

### BACKGROUND

Compositions have been designed to aid in promoting sleep. Such compositions are typically administered orally. It would be desirable to have a composition which could be administered topically that would aid in promoting sleep. It would be desirable to have a composition which could be delivered more directly than oral formulations to the areas of the brain which could be affected by such compositions.

### SUMMARY

Generally described, the present disclosure provides in a first exemplary embodiment a topically administered composition to promote sleep including Valeriana officinalis; alpha lipoic acid, at least one natural herb, Dead Sea minerals, and, a cream or gel carrier material suitable for topical application. Also disclosed is a method for treating sleeplessness comprising providing a formulation as described herein and administering an effective amount of the formulation to at least one of the scalp, forehead, temples and neck. Further disclosed is a method of making a homeopathic formulation to promote sleep, comprising providing a materials as described herein, blending the materials and blending the materials with a cream or gel.

Other features will become apparent upon reading the following detailed description of certain exemplary embodiments, when taken in conjunction with the appended claims.

### DETAILED DESCRIPTION

One aspect of the present disclosure provides a formulation for a topical cream applied, for example, to the scalp, forehead, temples and neck for transdermal delivery of the formulation to promote sleep. In one exemplary embodiment a formulation includes a combination of Valeriana officinalis (also known as valerian), natural herbs, Dead Sea minerals, alpha lipoic acid (an antioxidant), and other ingredients, all blended with a cream-based carrier for topical delivery. A number of the components, e.g., herbs, amino acids, and Dead Sea minerals, help relax and distress the mind and body, contributing to falling asleep and a relaxed sleep.

The natural herbs are one or more materials including chamomile germen (e.g., Chamomilla recutita flower extract), passion flower, Aloe barbadensis leaf juice, Valeriana officinal's root, Anthemis nobilis flower extract, Passiflora edulis flower extract, Lavandula officinalis, derivatives, combinations and mixtures of the foregoing, and the like.

The formulation may contain one or more oils, including palm oil (e.g., octyl palmitate), sesame seed oil, soybean oil, sweet almond oil, grapefruit peel oil, tea tree leaf oil, peppermint oil, sunflower oil, hydrogenated vegetable oil, derivatives, combinations and mixtures of the foregoing, and the like.

The formulation may contain one or more anti-oxidants, such as but not limited to alpha lipoic acid. The formulation may contain a cocktail of antioxidants, sometimes referred to as a “super antioxidant.”

The formulation may also contain 5-Hydroxytryptophan (5-HTP), Lactium, Melissa-lemon balm, and serotonin.
The carrier may be a cream, gel, or other carrier suitable for topical application.
In one exemplary embodiment a formulation includes:
Water
Octyl palmitate
Dead Sea Salt
Cetearyl alcohol
Glyceryl stearate
Glycerin
PEG-100 stearate
Sesamum indicum (sesame) seed oil
Dimothicone
Glycine soja (soybean)
Thiodic Acid
Prunus amygdalus dulcis (sweet almond) oil
Aloe barbadensis leaf juice
Chamomilla recutita (matricaria) flower extract
Eugenol
Camellia sinensis leaf extract
Tocopherol acetate
Ascorbic acid
Phytonsdione
Retinyl palmitate
Melatonin
Anthemis nobilis flower extract
Passiflora edulis flower extract
Tryptophan
Citrus grandis (grapefruit) peel oil
Vitis vinifera (grape) seed extract
Melalecua alemifolia (tea tree) leaf oil
Mentha piperita (peppermint) oil
Polysorbate 60
Thiamine
Steareth-2
Hydroxyethylcellulose
Disodium EDTA
Propylene Glycol
DMDM Hydantoin
Iodopropynyl butylcarbamate

In another exemplary embodiment, a formulation includes Piper Methysticum (Kava Kava) Root Extract, Valeriana Officinalis (Valerian) Root Extract, L-Theanine, Chamomilla Recutita (Matricaria) Flower Extract, Passiflora Edulis (Passion Flower) Extract, Cetyl Alcohol, Aloe Barbadensis Leaf Extract, Anthemis Nobilis (Roman Chamomile) Flower Extract, Camellia Sinensis (Green Tea) Extract, Vitis Vinifera (Grape) Seed Extract, Melatonin, Dimethicone, Vitis Vinifera (Grape) Seed Oil, Tocopheryl (Vitamin E) Acetate, Melaleuca Alternifolia (Tea Tree) Leaf Oil, Mentha Piperita (Peppermint) Leaf Oil, Ascorbic Acid (Vitamin C), Phytonadione (Vitamin K), Retinyl (Vitamin A) Palmitate, Cholecalciferol (Vitamin D), Thioctic (Alpha Lipoic) Acid, Thiamine (Vitamin B1), Dead Sea salt, and Eugenol, with the following ingredients included as the cream base: water, Propylene Glycol, Glyceryl Stearate SE, Glyceryl Stearate (and) PEG-100 Stearate, L-Tryptophan, Ethylhexyl Isononanoate, Caprylic/Capric Triglycerides, Hydrogenated Soybean Oil (and) Hydrogenated Cottonseed Oil, Stearic Acid, Cetearyl Alcohol (and) Ceteareth-20, Sesamum Indicum (Sesame) Seed Oil, Glycine Soja (Soybean) Oil, Prunus Amygdalus Dulcis (Sweet Almond) Oil, Aloe Barbadensis Leaf Juice, Xanthan Gum, Diazolidinyl Urea, DMDM Hydantoin.

In one aspect of the present disclosure the formulation may be applied by a user about 30 minutes before bedtime (or whenever sleep is desired). A feature of the formulation of the present disclosure is that topical application of the formulation allows for transdermal delivery of the formulation to the capillaries in the scalp and other areas proximate to the head and brain so as to promote faster delivery to the sleep centers of the brain than oral delivery vehicles can provide. Also, transdermal delivery avoids digestion and inactivation of important ingredients in the digestive tract as can occur with oral delivery.

The exemplary formulations have been shown in testing to promote sleep and aid in the treatment of insomnia and related ailments, based on anecdotal responses. The exemplary formulations provide a synergistic combination of components.

The following examples are set forth for purposes of illustration only. Parts and percentages appearing in such examples are by weight unless otherwise stipulated.

### EXAMPLES

### Example 1

The following formulation was prepared by mixing the ingredients: serotonin 5-HTP, valerian, melatonin, Melissa, lavender, lactium, tryptophan, chamomile, alpha lipoic acid, Dead Sea mineral salts, in a range from 3 mg to 1000 mg, with a cream base.

### Example 2

The following formulation was prepared by mixing the following ingredients with a cream base:
Kava Kava Extract: 6.4%
L-tryptophan: 2.8%
Valerian Root Extract: 1.62%
L-theanine: 1.62%
Melatonin: 0.6%
Alpha Lipoic Acid: 0.05%

Although only a number of exemplary embodiments have been described in detail above, those skilled in the art will readily appreciate that many modifications are possible in the exemplary embodiments without materially departing from the novel teachings and advantages. Accordingly, all such modifications are intended to be included within the scope of this disclosure as defined in the following claims.

While the methods, equipment and systems have been described in connection with specific embodiments, it is not intended that the scope be limited to the particular embodiments set forth, as the embodiments herein are intended in all respects to be illustrative rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect.

As used in the specification and the appended claims, the singular forms “a,” “an” and “the” include plural referents unless the context clearly dictates otherwise.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods, equipment and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods, equipment and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

It will be apparent to those skilled in the art that various modifications and variations can be made without departing from the scope or spirit. Other embodiments will be apparent to those skilled in the art from consideration of the specification and practice disclosed herein. It is intended that the specification and examples be considered as exemplary only, with a true scope and spirit being indicated by the following inventive concepts.

It should further be noted that any patents, applications and publications referred to herein are incorporated by reference in their entirety.

What is claimed is:

1. A homeopathic formulation to promote sleep, comprising:
a) Valeriana officinalis;
b) alpha lipoic acid;
c) at least one natural herb selected from the group consisting of chamomile germen, passion flower, Aloe barbadensis leaf juice, Valeriana officinalis root, Anthemis nobilis flower extract, Passiflora edulis flower extract, Lavandula officinalis, and mixtures thereof;
d) Dead Sea mineral salts; and,
e) a cream or gel carrier material suitable for topical application.

2. A homeopathic formulation to promote sleep, consisting essentially of:
a) Valeriana officinalis;
b) alpha lipoic acid;
c) at least one natural herb selected from the group consisting of chamomile germen, passion flower, Aloe barbadensis leaf juice, Valeriana officinal's root, Anthemis nobilis flower extract, Passiflora edulis flower extract, Lavandula officinalis, and mixtures thereof;
d) Dead Sea mineral salts; and,
e) a cream or gel carrier material suitable for topical application.

3. A method for treating sleeplessness, comprising:
a) providing a formulation according to claim 1; and,
b) administering an effective amount of the formulation of step a) to at least one of the scalp, forehead, temples and neck.

4. A method of making a homeopathic formulation to promote sleep, comprising:
a) providing a formulation according to claim 1;
b) blending the materials of step a); and,
c) blending the materials of step b) with a cream or gel.
